SEQUENCE LISTING (30 sequences)

SEQ ID NO: 1 - protein, 392 aa, Clostridium acetobutylicum
MKEVVIASAVRTAIGSYGKSLKDVPAVDLGATAIKEAVKKAGIKPEDVNEVILGNVLQAGLGQNPARQASFKAGLPVEIPAMTINKVCGSGLRTVSLAAQIIKAGDADVIIAGGMENMSRAPYLANNARWGYRMGNAKFVDEMITDGLWDAFNDYHMGITAENIAERWNISREEQDEFALASQKKAEEAIKSGQFKDEIVPVVIKGRKGETVVDTDEHPRFGSTIEGLAKLKPAFKKDGTVTAGNASGLNDCAAVLVIMSAEKAKELGVKPLAKIVSYGSAGVDPAIMGYGPFYATKAAIEKAGWTVDELDLIESNEAFAAQSLAVAKDLKFDMNKVNVNGGAIALGHPIGASGARILVTLVHAMQKRDAKKGLATLCIGGGQGTAILLEKC

SEQ ID NO: 2 - DNA, 1179 bp, Clostridium acetobutylicum
atgaaagaagttgtaatagctagtgcagtaagaacagcgattggatcttatggaaagtctcttaaggatgtaccagcagtagatttaggagctacagctataaaggaagcagttaaaaaagcaggaataaaaccagaggatgttaatgaagtcattttaggaaatgttcttcaagcaggtttaggacagaatccagcaagacaggcatcttttaaagcaggattaccagttgaaattccagctatgactattaataaggtttgtggttcaggacttagaacagttagcttagcagcacaaattataaaagcaggagatgctgacgtaataatagcaggtggtatggaaaatatgtctagagctccttacttagcgaataacgctagatggggatatagaatgggaaacgctaaatttgttgatgaaatgatcactgacggattgtgggatgcatttaatgattaccacatgggaataacagcagaaaacatagctgagagatggaacatttcaagagaagaacaagatgagtttgctcttgcatcacaaaaaaaagctgaagaagctataaaatcaggtcaatttaaagatgaaatagttcctgtagtaattaaaggcagaaagggagaaactgtagttgatacagatgagcaccctagatttggatcaactatagaaggacttgcaaaattaaaacctgccttcaaaaaagatggaacagttacagctggtaatgcatcaggattaaatgactgtgcagcagtacttgtaatcatgagtgcagaaaaagctaaagagcttggagtaaaaccacttgctaagatagtttcttatggttcagcaggagttgacccagcaataatgggatatggacctttctatgcaacaaaagcagctattgaaaaagcaggttggacagttgatgaattagatttaatagaatcaaatgaagcttttgcagctcaaagtttagcagtagcaaaagatttaaaatttgatatgaataaagtaaatgtaaatggaggagctattgcccttggtcatccaattggagcatcaggtgcaagaatactcgttactcttgtacacgcaatgcaaaaaagagatgcaaaaaaaggcttagcaactttatgtataggtggcggacaaggaacagcaatattgctagaaaagtgctag

SEQ ID NO: 3 - protein, 282 aa, Clostridium acetobutylicum
MKKVCVIGAGTMGSGIAQAFAAKGFEVVLRDIKDEFVDRGLDFINKNLSKLVKKGKIEEATKVEILTRISGTVDLNMAADCDLVIEAAVERMDIKKQIFADLDNICKPETILASNTSSLSITEVASATKRPDKVIGMHFFNPAPVMKLVEVIRGIATSQETFDAVKETSIAIGKDPVEVAEAPGFVVNRILIPMINEAVGILAEGIASVEDIDKAMKLGANHPMGPLELGDFIGLDICLAIMDVLYSETGDSKYRPHTLLKKYVRAGWLGRKSGKGFYDYSK

SEQ ID NO: 4 - DNA, 849 bp, Clostridium acetobultylicum
atgaaaaaggtatgtgttataggtgcaggtactatgggttcaggaattgctcaggcatttgcagctaaaggatttgaagtagtattaagagatattaaagatgaatttgttgatagaggattagattttatcaataaaaatctttctaaattagttaaaaaaggaaagatagaagaagctactaaagttgaaatcttaactagaatttccggaacagttgaccttaatatggcagctgattgcgatttagttatagaagcagctgttgaaagaatggatattaaaaagcagatttttgctgacttagacaatatatgcaagccagaaacaattcttgcatcaaatacatcatcactttcaataacagaagtggcatcagcaactaaaagacctgataaggttataggtatgcatttctttaatccagctcctgttatgaagcttgtagaggtaataagaggaatagctacatcacaagaaacttttgatgcagttaaagagacatctatagcaataggaaaagatcctgtagaagtagcagaagcaccaggatttgttgtaaatagaatattaataccaatgattaatgaagcagttggtatattagcagaaggaatagcttcagtagaagacatagataaagctatgaaacttggagctaatcacccaatgggaccattagaattaggtgattttataggtcttgatatatgtcttgctataatggatgttttatactcagaaactggagattctaagtatagaccacatacattacttaagaagtatgtaagagcaggatggcttggaagaaaatcaggaaaaggtttctacgattattcaaaataa

SEQ ID NO: 5 - protein, 261 aa, Clostrdium acetobutylicum
MELNNVILEKEGKVAVVTINRPKALNALNSDTLKEMDYVIGEIENDSEVLAVILTGAGEKSFVAGADISEMKEMNTIEGRKFGILGNKVFRRLELLEKPVIAAVNGFALGGGCEIAMSCDIRIASSNARFGQPEVGLGITPGFGGTQRLSRLVGMGMAKQLIFTAQNIKADEALRIGLVNKVVEPSELMNTAKEIANKIVSNAPVAVKLSKQAINRGMQCDIDTALAFESEAFGECFSTEDQKDAMTAFIEKRKIEGFKNR

SEQ ID NO: 6 - DNA, 786 bp, Clostridium acetobutylicum
atggaactaaacaatgtcatccttgaaaaggaaggtaaagttgctgtagttaccattaacagacctaaagcattaaatgcgttaaatagtgatacactaaaagaaatggattatgttataggtgaaattgaaaatgatagcgaagtacttgcagtaattttaactggagcaggagaaaaatcatttgtagcaggagcagatatttctgagatgaaggaaatgaataccattgaaggtagaaaattcgggatacttggaaataaagtgtttagaagattagaacttcttgaaaagcctgtaatagcagctgttaatggttttgctttaggaggcggatgcgaaatagctatgtcttgtgatataagaatagcttcaagcaacgcaagatttggtcaaccagaagtaggtctcggaataacacctggttttggtggtacacaaagactttcaagattagttggaatgggcatggcaaagcagcttatatttactgcacaaaatataaaggcagatgaagcattaagaatcggacttgtaaataaggtagtagaacctagtgaattaatgaatacagcaaaagaaattgcaaacaaaattgtgagcaatgctccagtagctgttaagttaagcaaacaggctattaatagaggaatgcagtgtgatattgatactgctttagcatttgaatcagaagcatttggagaatgcttttcaacagaggatcaaaaggatgcaatgacagctttcatagagaaaagaaaaattgaaggcttcaaaaatagatag

SEQ ID NO: 7 - protein, 379 aa, Clostridium acetobutlyicum
MDFNLTREQELVRQMVREFAENEVKPIAAEIDETERFPMENVKKMGQYGMMGIPFSKEYGGAGGDVLSYIIAVEELSKVCGTTGVILSAHTSLCASLINEHGTEEQKQKYLVPLAKGEKIGAYGLTEPNAGTDSGAQQTVAVLEGDHYVINGSKIFITNGGVADTFVIFAMTDRTKGTKGISAFIIEKGFKGFSIGKVEQKLGIRASSTTELVFEDMIVPVENMIGKEGKGFPIAMKTLDGGRIGIAAQALGIAEGAFNEARAYMKERKQFGRSLDKFQGLAWMMADMDVAIESARYLVYKAAYLKQAGLPYTVDAARAKLHAANVAMDVTTKAVQLFGGYGYTKDYPVERMMRDAKITEIYEGTSEVQKLVISGKIFR

SEQ ID NO: 8 - DNA, 1140 bp, Clostridium acetobutylicum
atggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataa

SEQ ID NO: 9 - protein, 858 aa, Clostridium acetobutylicum
MKVTNQKELKQKLNELREAQKKFATYTQEQVDKIFKQCAIAAAKERINLAKLAVEETGIGLVEDKIIKNHFAAEYIYNKYKNEKTCGIIDHDDSLGITKVAEPIGIVAAIVPTTNPTSTAIFKSLISLKTRNAIFFSPHPRAKKSTIAAAKLILDAAVKAGAPKNIIGWIDEPSIELSQDLMSEADIILATGGPSMVKAAYSSGKPAIGVGAGNTPAIIDESADIDMAVSSIILSKTYDNGVICASEQSILVMNSIYEKVKEEFVKRGSYILNQNEIAKIKETMFKNGAINADIVGKSAYIIAKMAGIEVPQTTKILIGEVQSVEKSELFSHEKLSPVLAMYKVKDFDEALKKAQRLIELGGSGHTSSLYIDSQNNKDKVKEFGLAMKTSRTFINMPSSQGASGDLYNFAIAPSFTLGCGTWGGNSVSQNVEPKHLLNIKSVAERRENMLWFKVPQKIYFKYGCLRFALKELKDMNKKRAFIVTDKDLFKLGYVNKITKVLDEIDIKYSIFTDIKSDPTIDSVKKGAKEMLNFEPDTIISIGGGSPMDAAKVMHLLYEYPEAEIENLAINFMDIRKRICNFPKLGTKAISVAIPTTAGTGSEATPFAVITNDETGMKYPLTSYELTPNMAIIDTELMLNMPRKLTAATGIDALVHAIEAYVSVMATDYTDELALRAIKMIFKYLPRAYKNGTNDIEAREKMAHASNIAGMAFANAFLGVCHSMAHKLGAMHHVPHGIACAVLIEEVIKYNATDCPTKQTAFPQYKSPNAKRKYAEIAEYLNLKGTSDTEKVTALIEAISKLKIDLSIPQNISAAGINKKDFYNTLDKMSELAFDDQCTTANPRYPLISELKDIYIKSF

SEQ ID NO: 10 - DNA, 2577 bp, Clostridium acetobutylicum
atgaaagttacaaatcaaaaagaactaaaacaaaagctaaatgaattgagagaagcgcaaaagaagtttgcaacctatactcaagagcaagttgataaaatttttaaacaatgtgccatagccgcagctaaagaaagaataaacttagctaaattagcagtagaagaaacaggaataggtcttgtagaagataaaattataaaaaatcattttgcagcagaatatatatacaataaatataaaaatgaaaaaacttgtggcataatagaccatgacgattctttaggcataacaaaggttgctgaaccaattggaattgttgcagccatagttcctactactaatccaacttccacagcaattttcaaatcattaatttctttaaaaacaagaaacgcaatattcttttcaccacatccacgtgcaaaaaaatctacaattgctgcagcaaaattaattttagatgcagctgttaaagcaggagcacctaaaaatataataggctggatagatgagccatcaatagaactttctcaagatttgatgagtgaagctgatataatattagcaacaggaggtccttcaatggttaaagcggcctattcatctggaaaacctgcaattggtgttggagcaggaaatacaccagcaataatagatgagagtgcagatatagatatggcagtaagctccataattttatcaaagacttatgacaatggagtaatatgcgcttctgaacaatcaatattagttatgaattcaatatacgaaaaagttaaagaggaatttgtaaaacgaggatcatatatactcaatcaaaatgaaatagctaaaataaaagaaactatgtttaaaaatggagctattaatgctgacatagttggaaaatctgcttatataattgctaaaatggcaggaattgaagttcctcaaactacaaagatacttataggcgaagtacaatctgttgaaaaaagcgagctgttctcacatgaaaaactatcaccagtacttgcaatgtataaagttaaggattttgatgaagctctaaaaaaggcacaaaggctaatagaattaggtggaagtggacacacgtcatctttatatatagattcacaaaacaataaggataaagttaaagaatttggattagcaatgaaaacttcaaggacatttattaacatgccttcttcacagggagcaagcggagatttatacaattttgcgatagcaccatcatttactcttggatgcggcacttggggaggaaactctgtatcgcaaaatgtagagcctaaacatttattaaatattaaaagtgttgctgaaagaagggaaaatatgctttggtttaaagtgccacaaaaaatatattttaaatatggatgtcttagatttgcattaaaagaattaaaagatatgaataagaaaagagcctttatagtaacagataaagatctttttaaacttggatatgttaataaaataacaaaggtactagatgagatagatattaaatacagtatatttacagatattaaatctgatccaactattgattcagtaaaaaaaggtgctaaagaaatgcttaactttgaacctgatactataatctctattggtggtggatcgccaatggatgcagcaaaggttatgcacttgttatatgaatatccagaagcagaaattgaaaatctagctataaactttatggatataagaaagagaatatgcaatttccctaaattaggtacaaaggcgatttcagtagctattcctacaactgctggtaccggttcagaggcaacaccttttgcagttataactaatgatgaaacaggaatgaaataccctttaacttcttatgaattgaccccaaacatggcaataatagatactgaattaatgttaaatatgcctagaaaattaacagcagcaactggaatagatgcattagttcatgctatagaagcatatgtttcggttatggctacggattatactgatgaattagccttaagagcaataaaaatgatatttaaatatttgcctagagcctataaaaatgggactaacgacattgaagcaagagaaaaaatggcacatgcctctaatattgcggggatggcatttgcaaatgctttcttaggtgtatgccattcaatggctcataaacttggggcaatgcatcacgttccacatggaattgcttgtgctgtattaatagaagaagttattaaatataacgctacagactgtccaacaaagcaaacagcattccctcaatataaatctcctaatgctaagagaaaatatgctgaaattgcagagtatttgaatttaaagggtactagcgataccgaaaaggtaacagccttaatagaagctatttcaaagttaaagatagatttgagtattccacaaaatataagtgccgctggaataaataaaaaagatttttataatacgctagataaaatgtcagagcttgcttttgatgaccaatgtacaacagctaatcctaggtatccacttataagtgaacttaaggatatctatataaaatcattttaa

SEQ ID NO: 11 - protein, 862 aa, Clostridium acetobutylicum
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 12 - DNA, 2589 bp, Clostridium acetobutylicum
atgaaagtcacaacagtaaaggaattagatgaaaaactcaaggtaattaaagaagctcaaaaaaaattctcttgttactcgcaagaaatggttgatgaaatctttagaaatgcagcaatggcagcaatcgacgcaaggatagagctagcaaaagcagctgttttggaaaccggtatgggcttagttgaagacaaggttataaaaaatcattttgcaggcgaatacatctataacaaatataaggatgaaaaaacctgcggtataattgaacgaaatgaaccctacggaattacaaaaatagcagaacctataggagttgtagctgctataatccctgtaacaaaccccacatcaacaacaatatttaaatccttaatatcccttaaaactagaaatggaattttcttttcgcctcacccaagggcaaaaaaatccacaatactagcagctaaaacaatacttgatgcagccgttaagagtggtgccccggaaaatataataggttggatagatgaaccttcaattgaactaactcaatatttaatgcaaaaagcagatataacccttgcaactggtggtccctcactagttaaatctgcttattcttccggaaaaccagcaataggtgttggtccgggtaacaccccagtaataattgatgaatctgctcatataaaaatggcagtaagttcaattatattatccaaaacctatgataatggtgttatatgtgcttctgaacaatctgtaatagtcttaaaatccatatataacaaggtaaaagatgagttccaagaaagaggagcttatataataaagaaaaacgaattggataaagtccgtgaagtgatttttaaagatggatccgtaaaccctaaaatagtcggacagtcagcttatactatagcagctatggctggcataaaagtacctaaaaccacaagaatattaataggagaagttacctccttaggtgaagaagaaccttttgcccacgaaaaactatctcctgttttggctatgtatgaggctgacaattttgatgatgctttaaaaaaagcagtaactctaataaacttaggaggcctcggccatacctcaggaatatatgcagatgaaataaaagcacgagataaaatagatagatttagtagtgccatgaaaaccgtaagaacctttgtaaatatcccaacctcacaaggtgcaagtggagatctatataattttagaataccaccttctttcacgcttggctgcggattttggggaggaaattctgtttccgagaatgttggtccaaaacatcttttgaatattaaaaccgtagctgaaaggagagaaaacatgctttggtttagagttccacataaagtatattttaagttcggttgtcttcaatttgctttaaaagatttaaaagatctaaagaaaaaaagagcctttatagttactgatagtgacccctataatttaaactatgttgattcaataataaaaatacttgagcacctagatattgattttaaagtatttaataaggttggaagagaagctgatcttaaaaccataaaaaaagcaactgaagaaatgtcctcctttatgccagacactataatagctttaggtggtacccctgaaatgagctctgcaaagctaatgtgggtactatatgaacatccagaagtaaaatttgaagatcttgcaataaaatttatggacataagaaagagaatatatactttcccaaaactcggtaaaaaggctatgttagttgcaattacaacttctgctggttccggttctgaggttactccttttgctttagtaactgacaataacactggaaataagtacatgttagcagattatgaaatgacaccaaatatggcaattgtagatgcagaacttatgatgaaaatgccaaagggattaaccgcttattcaggtatagatgcactagtaaatagtatagaagcatacacatccgtatatgcttcagaatacacaaacggactagcactagaggcaatacgattaatatttaaatatttgcctgaggcttacaaaaacggaagaaccaatgaaaaagcaagagagaaaatggctcacgcttcaactatggcaggtatggcatccgctaatgcatttctaggtctatgtcattccatggcaataaaattaagttcagaacacaatattcctagtggcattgccaatgcattactaatagaagaagtaataaaatttaacgcagttgataatcctgtaaaacaagccccttgcccacaatataagtatccaaacaccatatttagatatgctcgaattgcagattatataaagcttggaggaaatactgatgaggaaaaggtagatctcttaattaacaaaatacatgaactaaaaaaagctttaaatataccaacttcaataaaggatgcaggtgttttggaggaaaacttctattcctcccttgatagaatatctgaacttgcactagatgatcaatgcacaggcgctaatcctagatttcctcttacaagtgagataaaagaaatgtatataaattgttttaaaaaacaaccttaa

SEQ ID NO: 13 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 14 - DNA, 1170 bp, Clostridium acetobutylicum
atgctaagttttgattattcaataccaactaaagttttttttggaaaaggaaaaatagacgtaattggagaagaaattaagaaatatggctcaagagtgcttatagtttatggcggaggaagtataaaaaggaacggtatatatgatagagcaacagctatattaaaagaaaacaatatagctttctatgaactttcaggagtagagccaaatcctaggataacaacagtaaaaaaaggcatagaaatatgtagagaaaataatgtggatttagtattagcaatagggggaggaagtgcaatagactgttctaaggtaattgcagctggagtttattatgatggcgatacatgggacatggttaaagatccatctaaaataactaaagttcttccaattgcaagtatacttactctttcagcaacagggtctgaaatggatcaaattgcagtaatttcaaatatggagactaatgaaaagcttggagtaggacatgatgatatgagacctaaattttcagtgttagatcctacatatacttttacagtacctaaaaatcaaacagcagcgggaacagctgacattatgagtcacacctttgaatcttactttagtggtgttgaaggtgcttatgtgcaggacggtatagcagaagcaatcttaagaacatgtataaagtatggaaaaatagcaatggagaagactgatgattacgaggctagagctaatttgatgtgggcttcaagtttagctataaatggtctattatcacttggtaaggatagaaaatggagttgtcatcctatggaacacgagttaagtgcatattatgatataacacatggtgtaggacttgcaattttaacacctaattggatggaatatattctaaatgacgatacacttcataaatttgtttcttatggaataaatgtttggggaatagacaagaacaaagataactatgaaatagcacgagaggctattaaaaatacgagagaatactttaattcattgggtattccttcaaagcttagagaagttggaataggaaaagataaactagaactaatggcaaagcaagctgttagaaattctggaggaacaataggaagtttaagaccaataaatgcagaggatgttcttgagatatttaaaaaatcttattaa

SEQ ID NO: 15 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 16 - DNA, 1173 bp, Clostridium acetobutylicum
gtggttgatttcgaatattcaataccaactagaatttttttcggtaaagataagataaatgtacttggaagagagcttaaaaaatatggttctaaagtgcttatagtttatggtggaggaagtataaagagaaatggaatatatgataaagctgtaagtatacttgaaaaaaacagtattaaattttatgaacttgcaggagtagagccaaatccaagagtaactacagttgaaaaaggagttaaaatatgtagagaaaatggagttgaagtagtactagctataggtggaggaagtgcaatagattgcgcaaaggttatagcagcagcatgtgaatatgatggaaatccatgggatattgtgttagatggctcaaaaataaaaagggtgcttcctatagctagtatattaaccattgctgcaacaggatcagaaatggatacgtgggcagtaataaataatatggatacaaacgaaaaactaattgcggcacatccagatatggctcctaagttttctatattagatccaacgtatacgtataccgtacctaccaatcaaacagcagcaggaacagctgatattatgagtcatatatttgaggtgtattttagtaatacaaaaacagcatatttgcaggatagaatggcagaagcgttattaagaacttgtattaaatatggaggaatagctcttgagaagccggatgattatgaggcaagagccaatctaatgtgggcttcaagtcttgcgataaatggacttttaacatatggtaaagacactaattggagtgtacacttaatggaacatgaattaagtgcttattacgacataacacacggcgtagggcttgcaattttaacacctaattggatggagtatattttaaataatgatacagtgtacaagtttgttgaatatggtgtaaatgtttggggaatagacaaagaaaaaaatcactatgacatagcacatcaagcaatacaaaaaacaagagattactttgtaaatgtactaggtttaccatctagactgagagatgttggaattgaagaagaaaaattggacataatggcaaaggaatcagtaaagcttacaggaggaaccataggaaacctaagaccagtaaacgcctccgaagtcctacaaatattcaaaaaatctgtgtaa

SEQ ID NO: 17 - DNA, 1179 bp, Artificial Sequence
  Codon pair opt thil gene counterclockwise
ctaacacttttccaataagatggcagtaccttgaccaccaccgatacatagagtagccaaacccttcttggcatcacgcttttgcatagcgtggactaaagtaaccaagattctggcaccggaagcaccaattgggtgacccaaagcaatggcaccaccgttaacgttgaccttgttcatgtcgaatttcaagtccttggcaacagccaaagattgagcagcgaaagcttcgttggattcaatcaaatccaattcgtcaacggtccaaccagccttttcgatagcagccttggtagcgtagaaaggaccgtaacccatgatggctgggtcaacaccagcagaaccgtaggagacaatcttggccaatggcttgacacccaattccttggccttttcagcagacatgataaccaaaacagcagcacagtcgttcaaaccggaagcgttaccagcagtgacggtaccatccttcttgaaagctggtttcaacttggccaaaccttcaatggtggaaccgaatcttgggtgttcatcggtgtcgacaacggtttcaccctttctacccttgatgacaactgggacaatttcgtccttgaattgaccagatttgatggcttcttcagccttcttttgagaagccaaagcaaattcatcttgttcttctctggagatgttccatctttcagcaatgttttcagcagtgatacccatgtggtagtcgttgaaagcgtcccataaaccgtcagtgatcatttcatcgacgaacttggcgttacccattctgtaaccccatctagcattgttagccaagtatggagctctggacatgttttccataccaccagcaatgatgacatcagcgtcaccagccttgatgatttgagcagccaaagaaacagttctcaaaccagaaccacaaaccttgttgatggtcatggctggaatttcaactggcaaaccagccttgaaagaagcttgacgagctgggttttgacctaaaccagcttgcaaaacgttacctaagataacttcgttaacatcttctggcttgataccagccttcttgacagcttccttgatggcggtagcacccaagtcgacagctgggacgtccttcaaagacttaccgtaagaaccaatggcagttctgacagcagaagcaataacaacttccttcat

SEQ ID NO: 18 - DNA, 850 bp, Artificial Sequence
  Codon pair opt hbd gene
catgaagaaggtttgtgtcattggtgccggtaccatgggttctggtattgctcaagctttcgctgccaagggtttcgaagttgttttgagagatatcaaggacgaattcgttgaccgtggtttggatttcatcaacaagaacttgtccaagttggtcaagaagggtaagattgaagaagctaccaaggtcgaaatcttgaccagaatctccggtactgttgacttgaacatggctgctgactgtgatttggtcattgaagctgccgttgaaagaatggacatcaagaagcaaatctttgctgatttggacaacatctgtaagccagaaaccattttggcttccaacacttcttctttgtccatcactgaagttgcttctgctaccaagagaccagacaaggttatcggtatgcacttcttcaacccagctccagtcatgaagttggtcgaagtcatcagaggtattgccacctctcaagaaactttcgatgctgtcaaggaaacttccattgccattggtaaggacccagttgaagttgctgaagctccaggtttcgttgtcaacagaatcttgattccaatgatcaacgaagctgtcggtattttggctgaaggtattgcttctgttgaagatatcgacaaggccatgaaattgggtgctaaccacccaatgggtccattggaattaggtgacttcatcggtttggatatctgtttggccatcatggatgtcttatactctgaaaccggtgactctaagtacagacctcacactttattgaagaagtacgttagagctggttggttaggtagaaagtctggtaagggtttctacgactactccaaatag

SEQ ID NO: 19 - DNA, 789 bp, Artificial Sequence
  Codon pair opt crt gene counterclockwise
tcatcacctgttcttgaaaccttcaatctttctcttttcgatgaaagcggtcatagcatccttttggtcttcagtggagaaacattcaccgaaagcttcagattcaaaggccaaagcggtgtcgatatcacattgcatacctctgttgatggcttgcttggacaatttgacagcaactggagcgttggagacgatcttgttagcaatttccttggcagtgttcatcaattcagatggttcaacaaccttgttgactaaaccaattctcaaagcttcgtcagccttgatgttttgagcggtgaagatcaattgcttggccatacccataccaaccaatctggataatctttgagtaccaccgaaacctggagtgatacctagaccgacttctggttgaccgaaacgagcgttagaagaagcaattctgatgtcacaggacatggcaatttcacaaccaccacccaaagcgaaaccgttgacagcagcaatgactggcttttccaacaattccaatcttctgaaaaccttgttacctaagataccgaactttctaccttcaatggtgttcatttccttcatttcagagatatcagcaccagcaacgaaagacttttcaccggcaccggtcaagatgacagccaaaacttcagaatcgttttcaatttcaccaatgacgtagtccatttccttcaaagtgtcagagttcaaagcattcaaagcctttggtctgttgatggtgacaacggcaaccttaccttccttttccaagataacgttgttcaattccat

SEQ ID NO: 20 - DNA, 1141 bp, Artificial Sequence
  Codon pair opt bcd gene
catggacttcaacttgaccagagaacaagaattggtcagacaaatggttagagaatttgctgaaaacgaagttaagccaattgctgctgaaatcgatgaaactgaaagattcccaatggaaaacgtcaagaagatgggtcaatacggtatgatgggtattccattctctaaggaatacggtggtgctggtggtgacgtcttgtcttacatcattgctgtcgaagaattgtccaaggtttgtggtaccactggtgtcatcttatctgctcacacttctctatgtgcctccttgatcaacgaacacggtactgaagaacaaaagcaaaagtacttggttccattggccaagggtgaaaagattggtgcctacggtttgactgaaccaaacgctggtactgactctggtgctcaacaaactgttgccgttttggaaggtgaccactacgtcatcaacggttccaagatcttcatcaccaacggtggtgttgctgacacctttgtcatcttcgctatgaccgatcgtaccaagggtaccaagggtatctctgctttcattattgaaaagggtttcaagggtttctccatcggtaaggtcgaacaaaagttgggtatcagagcttcctctaccactgaattggttttcgaagacatgattgttccagttgaaaacatgatcggtaaggaaggtaagggtttcccaattgccatgaagactttagatggtggtagaattggtattgctgctcaagctttgggtattgctgaaggtgccttcaacgaagctagagcttacatgaaggaaagaaagcaattcggtagatctttggacaaattccaaggtttggcttggatgatggctgacatggacgttgccatcgaatctgctcgttacttggtctacaaggctgcttacttgaagcaagctggtttgccatacaccgtcgatgctgccagagctaagttgcacgctgccaacgttgccatggatgtcaccaccaaggctgtccaattattcggtggttacggttacaccaaggactacccagttgaaagaatgatgagagatgctaagatcactgaaatctacgaaggtacttctgaagttcaaaagttggttatctccggtaagatcttcagatag

SEQ ID NO: 21 - DNA, 2577 bp, Artificial Sequence
  Codon pair opt adhE gene
atgaaggttaccaaccaaaaggaattgaagcaaaagttgaacgaattgagagaagctcaaaagaagttcgctacctacactcaagaacaagttgacaagatcttcaagcaatgtgccattgctgctgccaaggaacgtatcaacttggccaagttggctgtcgaagaaaccggtattggtttggttgaagacaagatcatcaagaaccacttcgctgctgaatacatctacaacaagtacaagaacgaaaagacctgtggtatcatcgaccacgatgactctttgggtatcaccaaggttgctgaaccaatcggtattgtcgccgccattgtcccaaccactaacccaacttccactgccatcttcaaatctttgatctccttgaagaccagaaacgctatcttcttctccccacacccaagagccaagaagtccaccattgctgctgccaaattaatcttggatgctgctgttaaggctggtgccccaaagaacattattggttggatcgatgaaccttccattgaattgtctcaagacttgatgtctgaagctgatatcatcttggctaccggtggtccatccatggtcaaggccgcttactcttctggtaagccagctattggtgttggtgctggtaacactccagctatcatcgatgaatctgctgacattgacatggctgtctcctccattatcttgtccaagacttatgacaacggtgtcatctgtgcctctgaacaatccatcttggttatgaactctatctacgaaaaggtcaaggaagaatttgttaagagaggttcctacatcttaaaccaaaatgaaattgccaagatcaaggaaaccatgttcaagaacggtgccatcaacgctgacattgtcggtaaatctgcttacatcattgccaagatggctggtattgaagttccacaaaccactaagattttgatcggtgaagttcaatctgtcgaaaagtctgaattattctctcacgaaaagttgtctccagtcttggctatgtacaaggtcaaggatttcgacgaagctttgaagaaggctcaaagattaattgaattaggtggttctggtcacacctcttctctatacattgactctcaaaacaacaaggacaaggtcaaggaattcggtctagctatgaagacttccagaactttcatcaacatgccatcttctcaaggtgcttctggtgatttgtacaactttgccattgctccatctttcactttaggttgtggtacctggggtggtaactctgtttctcaaaacgttgaaccaaagcatttgctaaacatcaagtccgttgctgaaagaagagaaaacatgttgtggttcaaggttccacaaaagatctacttcaaatacggttgtttgagatttgctttgaaggaattgaaagatatgaacaagaagcgtgctttcatcgttactgacaaggatttgttcaaattgggttacgttaacaagatcactaaggttttggatgaaattgatatcaagtactccatcttcactgatatcaaatctgacccaaccattgactccgtcaagaagggtgctaaggaaatgttgaacttcgaaccagataccattatctccattggtggtggttctccaatggatgctgccaaggttatgcatttgttgtacgaatacccagaagctgaaatcgaaaacttggccatcaacttcatggacatcagaaagagaatctgtaacttcccaaagttgggtaccaaggccatttctgttgccattccaaccaccgctggtaccggttctgaagctactccatttgctgtcatcaccaacgacgaaaccggtatgaagtacccattgacctcttacgaattgactccaaacatggccatcattgacactgaattgatgttgaacatgccaagaaagttgactgctgctaccggtattgacgctttagtccacgctatcgaagcttacgtctccgttatggccactgactacactgacgaattggctttgagagctatcaagatgatcttcaagtacttgccaagagcttacaagaacggtactaacgatatcgaagctcgtgaaaagatggctcacgcttccaacattgctggtatggctttcgctaacgctttcttgggtgtttgtcactccatggcccacaagttgggtgctatgcaccacgttcctcacggtattgcttgtgctgttttgattgaagaagtcatcaagtacaacgctactgactgtccaaccaagcaaactgctttcccacaatacaagtctccaaacgccaagagaaagtacgctgaaattgctgaatacttgaacttgaaaggtacttctgacactgaaaaggtcactgctttaatcgaagctatctccaagttgaagattgacttatctattcctcaaaacatctctgctgctggtattaacaagaaggacttctacaacactttagacaagatgtccgaattggctttcgatgaccaatgtaccaccgctaacccaagatacccattgatctctgaattgaaggatatctacatcaagtccttttaa

SEQ ID NO: 22 - DNA, 2589 bp, Artificial Sequence
  Codon pair opt adhE1 gene
atgaaggtcaccactgtcaaggaattggatgaaaagttgaaggtcatcaaggaagctcaaaagaagttctcttgttactctcaagaaatggttgacgaaatcttcagaaacgctgctatggctgccattgacgccagaattgaattggccaaggctgccgtcttggaaaccggtatgggtttggttgaagacaaggttatcaagaaccacttcgctggtgaatacatctacaacaaatacaaggatgaaaagacttgtggtatcatcgaaagaaacgaaccatacggtatcaccaagattgctgaacctatcggtgtcgttgctgccatcatcccagttaccaacccaacttccaccaccattttcaaatccttgatctctttgaagaccagaaacggtattttcttctctcctcacccaagagctaagaagtccactatcttagctgccaagactatcttagatgctgctgtcaagtctggtgctccagaaaacattattggttggattgacgaaccatccattgaattgactcaatacttgatgcaaaaggctgatatcactttggccactggtggtccatctttggtcaagtctgcttactcctctggtaagccagctattggtgtcggtccaggtaacactccagtcatcattgatgaatctgctcacatcaagatggctgtctcctctatcatcttgtccaagacttacgacaacggtgttatctgtgcttctgaacaatctgttatcgttttgaaatccatctacaacaaggtcaaggacgaattccaagaaagaggtgcttacatcatcaagaagaacgaattggacaaggtcagagaagtcattttcaaggacggttccgttaacccaaagattgttggtcaatctgcctacaccattgctgctatggctggtatcaaggttccaaagaccaccagaatcttgattggtgaagtcacctctttaggtgaagaagaaccatttgctcacgaaaaattgtctccagttttggccatgtacgaagctgacaactttgacgatgctttgaagaaggctgttaccttaatcaacttaggtggtttgggtcacacttctggtatctacgctgatgaaatcaaggcccgtgacaagattgacagattctcctctgctatgaagaccgttagaacctttgttaacattccaacttcccaaggtgcttctggtgatttgtacaacttcagaattccaccttctttcactttaggttgtggtttctggggtggtaactccgtttctgaaaacgttggtccaaagcatttgttgaacatcaagactgttgctgaaagaagagaaaacatgttatggttccgtgttccacacaaggtctacttcaagttcggttgtttgcaatttgctttgaaggacttaaaggatttgaagaagaagagagctttcattgtcactgactccgatccttacaacttgaactacgttgactctatcatcaagatcttggaacacttggatatcgatttcaaggtcttcaacaaggttggtagagaagctgatttgaagactatcaagaaggctactgaagaaatgtcctctttcatgccagacactatcattgctttaggtggtaccccagaaatgtcttctgccaagttgatgtgggttttgtacgaacatccagaagttaagttcgaagacttggccatcaagttcatggacatcagaaagagaatctacactttcccaaagttgggtaagaaggctatgttggttgccattaccacctctgctggttctggttctgaagtcactccatttgctttggttaccgacaacaacaccggtaacaaatacatgttggctgactacgaaatgaccccaaacatggccattgtcgacgctgaattgatgatgaagatgccaaagggtttgactgcttactccggtatcgatgctttggtcaactctattgaagcttacacttccgtttacgcctccgaatacaccaacggtttggctttggaagccatcagattaatcttcaagtacttgccagaagcttacaagaacggtcgtaccaatgaaaaggctagagaaaagatggctcacgcttccaccatggctggtatggcttccgctaacgctttcttaggtttgtgtcactccatggccatcaaattgtcctctgaacacaacattccatccggtattgccaatgctttgttgattgaagaagtcatcaagttcaatgctgttgacaacccagtcaagcaagctccatgtccacaatacaagtacccaaacaccattttccgttacgccagaattgctgactacatcaagttgggtggtaacactgacgaagaaaaggtcgatttattaatcaacaagattcacgaattgaagaaggctttgaacattccaacttctatcaaggatgctggtgttttggaagaaaacttctactcttctttggacagaatctctgaattggccttggatgaccaatgtaccggtgctaacccaagattcccattgacttctgaaatcaaggaaatgtacattaactgtttcaagaagcaaccatag

SEQ ID NO: 23 - DNA, 1170 bp, Artificial Sequence
  Codon pair opt bdhA gene
atgttgtctttcgactactccattccaactaaggttttcttcggtaagggtaagatcgatgttatcggtgaagaaatcaagaaatacggttccagagttttgattgtctacggtggtggttccatcaagagaaacggtatctacgatcgtgccactgccatcttaaaggaaaacaacattgctttctacgaattatctggtgttgaaccaaacccaagaatcactaccgtcaagaagggtattgaaatctgtagagaaaacaacgttgacttggtcttggccattggtggtggttctgctattgactgttccaaggtcattgctgctggtgtttactacgatggtgacacctgggacatggttaaggacccttccaagatcaccaaggttttgccaattgcttccatcttgactttatctgctaccggttctgaaatggaccaaatcgccgtcatctccaacatggaaaccaatgaaaagttgggtgttggtcacgatgacatgagaccaaagttctctgtcttggacccaacctacactttcaccgttccaaagaaccaaactgctgctggtactgctgatatcatgtctcacactttcgaatcttacttctctggtgtcgaaggtgcttacgttcaagatggtattgctgaagctatcttgagaacctgtatcaaatacggtaagattgctatggaaaagaccgatgactacgaagctagagctaacttgatgtgggcttcttccttggctatcaacggtttattgtctttaggtaaggacagaaagtggtcctgtcacccaatggaacacgaattgtctgcttactacgatatcactcacggtgttggtttggccatcttgactccaaactggatggaatacatcttgaacgatgacactttgcacaagtttgtctcctacggtatcaacgtctggggtattgacaagaacaaggacaactacgaaattgccagagaagctatcaagaacaccagagaatacttcaactctttgggtattccatccaagttgcgtgaagtcggtattggtaaggacaaattggaattgatggccaagcaagctgtcagaaactctggtggtaccattggttctttgagaccaatcaatgctgaagatgttttggaaatcttcaagaagtcttactaa

SEQ ID NO: 24 - DNA, 1173 bp, Artificial Sequence
  Codon pair opt bdhB gene
atggtcgatttcgaatactctatcccaaccagaatcttcttcggtaaggacaagatcaacgttttgggtagagaattgaagaaatacggttccaaggttttgattgtctacggtggtggttccatcaagagaaacggtatctacgacaaggctgtctccattttggaaaagaactctatcaaattctacgaattggctggtgttgaaccaaacccaagagttaccaccgtcgaaaagggtgtcaagatctgtcgtgaaaacggtgttgaagttgttttggccatcggtggtggttctgccattgactgtgccaaggtcattgctgctgcctgtgaatacgatggtaacccatgggacattgtcttggatggttctaagatcaagcgtgtcttaccaattgcttccatcttgactatcgctgctactggttctgaaatggacacctgggctgttatcaacaacatggacactaacgaaaagttgattgctgctcacccagatatggccccaaagttctctattttggacccaacctacacttacactgttccaaccaaccaaactgctgctggtactgctgatatcatgtctcacatctttgaagtttacttctccaacaccaagaccgcttacttgcaagacagaatggctgaagctctattaagaacctgtatcaagtacggtggtattgctttggaaaagccagatgactacgaagccagagctaacttgatgtgggcttcctctttggctatcaacggtttattgacttacggtaaggacaccaactggtccgttcatttgatggaacacgaattgtctgcttactacgatatcactcacggtgtcggtttggccatcttgactccaaactggatggaatacattttgaacaacgacactgtctacaagttcgtcgaatacggtgttaacgtctggggtattgacaaggaaaagaaccactacgacattgctcaccaagccatccaaaagaccagagactatttcgtcaacgttttgggtttaccatccagattaagagatgttggtattgaagaagaaaaattggatatcatggctaaggaatctgtcaaattgactggtggtaccattggtaacttgagacctgttaacgcttctgaagttttgcaaatcttcaagaaatctgtttag

SEQ ID NO: 25 - DNA, 290 bp, Artificial Sequence
  Promoter Gal7
tccctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 26 - DNA, 348 bp, Artificial Sequence
  Terminator Gal7
aaagaaagtggaatattcattcatatcatattttttctattaactgcctggtttcttttaaattttttattggttgtcgacttgaacggagtgacaatatatatatatatatatttaataatgacatcattatctgtaaatctgattcttaatgctattctagttatgtaagagtggtcctttccataaaaaaaaaaaaaaagaaaaaagaattttaggaatacaatgcagcttgtaagtaaaatctggaatattcatatcgccacaacttcttatgcttataaaagcactaatgcctgaatttatgttgaaaatatgtgtcacaaataaagaaactgtgacatctgg

SEQ ID NO: 27 - DNA, 356 bp, Artificial Sequence
  Terminator Gal10 counterclockwise
cgcgcccaataatatttacaacttttccttatgattttttcactgaagcgcttcgcaatagttgtgagtgatatcaaaagtaacgaaatgaactccgcggctcgtgctatattcttgttgctaccgtccatatctttccatagattttcaatttttgatgtctccatggtggtacagagaacttgtaaacaattcggtccctacatgtgaggaaattcgctgtgacacttttatcactgaactccaaatttaaaaaatagcataaaattcgttatacagcaaatctatgtgttgcaattaagaactaaaagatatagagtgcatattttcaagaaggatagtaagctggcaaatca

SEQ ID NO: 28 - DNA, 336 bp, Artificial Sequence
  Promoter Gal10 counterclockwise
ttatattgaattttcaaaaattcttactttttttttggatggacgcaaagaagtttaataatcatattacatggcattaccaccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggcgacagccctccgacggaagactctcctccgtgcgtcctcgtcttcaccggtcgcgttcctgaaacgcagatgtgcctcgcgccgcactgctccgaac

SEQ ID NO: 29 - DNA, 300 bp, Artificial Sequence
  Promoter Gal1
aataaagattctacaatactagcttttatggttatgaagaggaaaaattggcagtaacctggccccacaaaccttcaaattaacgaatcaaattaacaaccataggatgataatgcgattagttttttagccttatttctggggtaattaatcagcgaagcgatgatttttgatctattaacagatatataaatggaaaagctgcataaccactttaactaatactttcaacattttcagtttgtattacttcttattcaaatgtcataaaagtatcaacaaaaaattgttaatatacct

SEQ ID NO: 30 - DNA, 347 bp, Artificial Sequence
  Terminator Gal1
gtatacttcttttttttactttgttcagaacaacttctcatttttttctactcataactttagcatcacaaaatacgcaataataacgagtagtaacacttttatagttcatacatgcttcaactacttaataaatgattgtatgataatgttttcaatgtaagagatttcgattatccacaaactttaaaacacagggacaaaattcttgatatgctttcaaccgctgcgttttggatacctattcttgacatgatatgactaccattttgttattgtacgtggggcagttgacgtcttatcatatgtcaaagtcatttgcgaagttcttggcaagttgccaactg

(19) United States
(12) Patent Application Publication
(10) Pub. No.: US 2010/0036174 A1
(43) Pub. Date: Feb. 11, 2010

(54) BUTANOL PRODUCTION IN A EUKARYOTIC CELL

(76) Inventors: Lourina Madeleine Raamsdonk, Den Haag (NL); Wilhelmus Theodorus Antonius Maria De Laat, Breda (NL); Marco Alexander Van Den Berg, Poeldijk (NL)

Correspondence Address: NIXON & VANDERHYE, PC, 901 NORTH GLEBE ROAD, 11TH FLOOR, ARLINGTON, VA, 22203, US

(21) Appl. No.: 12/447,740

(22) PCT Filed: Oct. 30, 2007
(86) PCT No.: PCT/EP2007/061685

§ 371 Date: Apr. 29, 2009

Related U.S. Application Data
(60) Provisional application No. 60/855,370, filed on Oct. 31, 2006.
(60) Provisional application No. 60/935,029, filed on Jul. 23, 2007.

(30) Foreign Application Priority Data
Oct. 31, 2006 (EP) 06123259.1
Jul. 23, 2007 (EP) 07112954.8

(51) Int. Cl. C12P 7/16; C12N 1/16; C07C 31/12

(52) U.S. Cl. 568/840; 435/254.21; 435/160

(57) ABSTRACT

The present invention relates to a transformed eukaryotic cell comprising one or more nucleotide sequence(s) encoding acetyl-CoA acetyltransferase, 3-hydroxybutyryl-CoA dehydrogenase, 3-hydroxybutyryl-CoA dehydratase, butyryl-CoA dehydrogenase, alcohol dehydrogenase or acetaldehyde dehydrogenase and/or NAD(P)H-dependent butanol dehydrogenase, whereby the nucleotide sequence(s) upon transformation of the cell confer(s) the cell the ability to produce butanol. The invention also relates to a process for the production of butanol.

The present invention relates to a transformed eukaryotic cell capable of producing butanol and a process for the production of butanol by using the transformed eukaryotic cell.

The acetone/butanol/ethanol (ABE) fermentation process has received considerable attention in the recent years as a prospective process for the production of commodity chemicals, such as butanol and acetone from biomass.

The fermentation of carbohydrates to acetone, butanol, and ethanol by solventogenic Clostridia is well known since decades. Clostridia produce butanol by conversion of a suitable carbon source into acetyl-CoA. Substrate acetyl-CoA then enters into the solventogenesis pathway to produce butanol using six concerted enzyme reactions. The reactions and enzymes catalysing these reactions are listed below:

| | |
|---|---|
| 2 Acetyl-CoA → Acetoacetyl-CoA + HSCoA | (acetyl-CoA acetyl transferase) |
| Acetoacetyl-CoA + NAD(P)H → 3-hydroxylbutyryl-CoA + NAD(P)⁺ | (3-hydroxyl-CoA dehydrogenase) |
| 3-hydroxylbutyryl-CoA → Crotonyl-CoA + H₂O | (3-hydroxybutyryl-CoA dehydratase) |
| Crotonyl-CoA + NAD(P)H → Butyryl-CoA + NAD(P)⁺ | (butyryl-CoA dehydrogenase) |
| Butyryl-CoA + NAD(P)H → Butyraldehyde + CoA + NAD(P)⁺ | (butyraldehyde dehydrogenase) |
| Butyraldehyde + NAD(P)H → Butanol + NAD(P)⁺ | (butanol dehydrogenase) |

The formation of butanol requires the conversion of acetyl-CoA into acetoacetyl-CoA by acetyl transferase. This reaction is followed by the conversion of acetoacetyl-CoA into 3-hydroxylbutyryl-CoA by 3-hydroxyl-CoA dehydrogenase, which is followed by the conversion of 3-hydroxylbutyryl-CoA into crotonyl-CoA by 3-hydroxybutyryl-CoA dehydratase (also named crotonase) and the conversion of crotonyl-CoA into butyryl-CoA by butyryl-CoA dehydrogenase and followed by the conversion of butyryl-CoA to butyraldehyde by butyraldehyde dehydrogenase, with the final conversion of butyrylaldehyde to butanol by butanol dehydrogenase (Jones, D. T., Woods, D. R., 1986, Microbiol. Rev., 50, 484-524).

However, the production of butanol suffers from poor process economics, because the butanol produced is toxic for the microbial cells and thus titers are low. Many studies have been directed to increase the resistance of Clostridia strains against butanol and consequently achieve an increase in titers. In U.S. Pat. No. 6,960,465, it is for instance shown that overexpression of the heat shock proteins in Clostridium acetobutylicum resulted in an increased butanol production yield compared to the wild type strain.

Since Clostridia are sensitive to oxygen, Clostridia-fermentations need to be operated under strict anaerobic conditions, which makes it difficult to operate such fermentations on a large scale. Anaerobic fermentations generally result in low biomass concentrations due to the low ATP-gain under anaerobic conditions. In addition, Clostridia are sensitive to bacteriophages, causing lysis of the bacterial cells during fermentation. Since Clostridia fermentations are carried out at neutral pH, sterile conditions are essential to prevent contamination of the fermentation broth by eg. lactic acid bacteria, which lead to high costs for fermentations on an industrial scale (Zverlov et al. Appl. Microbiol. Biotechnol. Vol. 71, p. 587-597, 2006, Spivey, Process Biochemistry November 1978). Another disadvantage of butanol production in Clostridia is that undesirable by-products like, acetone, acetate and butyrate are also produced, which lowers the yield of butanol on carbon.

WO2007/041269 discloses a recombinant microorganism, for instance a yeast such as Saccharomyces cerevisiae, which is transformed with at least one DNA molecule encoding a polypeptide that catalyses one of the reactions of the butanol pathway as described above. However, the amount of butanol produced in a genetically modified Saccharomyces strain disclosed in WO 2007/041269 was only between 0.2 to 1.7 mg/l, which is a factor of about 10,000-100,000 lower than the amount of butanol produced in a classical ABE fermentation.

The aim of the present invention is the provision of an alternative eukaryotic cell capable of producing a higher amount of butanol than known in the state of the art.

The aim is achieved according to the invention with a transformed eukaryotic cell comprising one or more nucleotide sequence(s) encoding acetyl-CoA acetyltransferase, 3-hydroxybutyryl-CoA dehydrogenase, 3-hydroxybutyryl-CoA dehydratase, butyryl-CoA dehydrogenase, alcohol dehydrogenase or acetaldehyde dehydrogenase and/or NAD(P)H-dependent butanol dehydrogenase whereby the nucleotide sequence(s) upon transformation of the cell confers the cell the ability to produce butanol.

The invention also relates to a transformed eukaryotic cell selected from the group consisting of Aspergillus, Penicillium, Pichia, Kluyveromyces, Yarrowia, Candida, Hansenula, Humicola, Torulaspora, Trichosporon, Brettanomyces, Zygosaccharomyces, Pachysolen and Yamadazyma, comprising one or more nucleotide sequence(s) encoding acetyl-CoA acetyltransferase, 3-hydroxybutyryl-CoA dehydrogenase, 3-hydroxybutyryl-CoA dehydratase, butyryl-CoA dehydrogenase, alcohol dehydrogenase or acetaldehyde dehydrogenase and/or NAD(P)H-dependent butanol dehydrogenase whereby the nucleotide sequence(s) upon transformation of the cell confers the cell the ability to produce butanol.

As used herein a transformed eukaryotic cell is defined as a eukaryotic cell which is genetically modified or transformed with one or more of the nucleotide sequences as defined herein. A eukaryotic cell that is not transformed or genetically modified, is a cell which does not comprise one or more of the nucleotide sequences enabling the cell tyo produce butanol. Hence, a non-transformed eukaryotic cell is a cell that does not naturally produce butanol. As used herein, butanol is n-butanol or 1-butanol.

In the scope of the present invention, alcohol dehydrogenase or acetaldehyde dehydrogenase catalyses the same reaction as butyraldehyde dehydrogenase. The alcohol dehydrogenase or acetaldehyde dehydrogenase in the present invention may also have butanol dehydrogenase activity.

Preferably, the eukaryotic cell according to the present invention expresses one or more nucleotide sequence(s) selected from the group consisting of:
a. a nucleotide sequence encoding an acetyl-CoA acetyltransferase, wherein said nucleotide sequence is selected from the group consisting of:
   i. a nucleotide sequence encoding an acetyl-CoA acetyltransferase comprising an amino acid sequence that has at least 20%, preferably at least 25, 30, 40, 50, 55, 60, 65, 70, 75, 80, 85, 90, 95, 96, 97, 98, or 99% sequence identity with the amino acid sequence of SEQ ID NO: 1,
   ii. a nucleotide sequence that has at least 15%, preferably at least 20, 25, 30, 40, 50, 60, 70, 80, 90, 95, 96, 97, 98, 99% sequence identity with the nucleotide sequence of SEQ ID NO:2.
   iii. a nucleotide sequence the complementary strand of which hybridizes to a nucleic acid molecule of sequence of (i) or (ii); and
   iv. a nucleotide sequence which differs from the sequence of a nucleic acid molecule of (iii) due to the degeneracy of the genetic code,
b. a nucleotide sequence encoding an a 3-hydroxybutyryl-CoA dehydrogenase, wherein said nucleotide sequence is selected from the group consisting of:
   i. a nucleotide sequence encoding a 3-hydroxybutyryl-CoA dehydrogenase, said 3-hydroxybutyryl-CoA dehydrogenase comprising an amino acid sequence that has at least 25%, preferably at least 30, 40, 50, 55, 60, 65, 70, 75, 80, 85, 90, 95, 96, 97, 98, 99% sequence identity with the amino acid sequence of SEQ ID NO: 3,
   ii. a nucleotide sequence that has at least 20% preferably at least 25, 30, 40, 50, 55, 60, 65, 70, 75, 80, 85, 90, 95, 96, 97, 98, 99% sequence identity with the nucleotide sequence of SEQ ID NO:4,
   iii. a nucleotide sequence the complementary strand of which hybridizes to a nucleic acid molecule of sequence of (i) or (ii); and,
   iv. a nucleotide sequence which differs from the sequence of a nucleic acid molecule of (iii) due to the degeneracy of the genetic code,
c. a nucleotide sequence encoding 3-hydroxybutyryl-CoA dehydratase, wherein said nucleotide sequence is selected from the group consisting of:
   i. a nucleotide sequence encoding a 3-hydroxybutyryl-CoA dehydratase, said 3-hydroxybutyryl-CoA dehydratase comprising an amino acid sequence that has at least 30%, preferably at least 40, 50, 55, 60, 65, 70, 75, 80, 85, 90, 95, 96, 97, 98, 99% sequence identity with the amino acid sequence of SEQ ID NO: 5;
   ii. a nucleotide sequence comprising a nucleotide sequence that has at least 25%, preferably at least 30, 40, 50, 55, 60, 65, 70, 75, 80, 85, 90, 95, 96, 97, 98, 99% sequence identity with the nucleotide sequence of SEQ ID NO:6;
   iii. a nucleotide sequence the complementary strand of which hybridizes to a nucleic acid molecule of sequence of (i) or (ii); and
   iv. a nucleotide sequence which differs from the sequence of a nucleic acid molecule of (iii) due to the degeneracy of the genetic code,
d. a nucleotide sequence encoding butyryl-CoA dehydrogenase, wherein said nucleotide sequence is selected from the group consisting of:
   i. a nucleotide sequence encoding a butyryl-CoA dehydrogenase, said butyryl-CoA dehydrogenase comprising an amino acid sequence that has at least 20%, preferably at least 25, 30, 40, 50, 55, 60, 65, 70, 75, 80, 85, 90, 95, 96, 97, 98, 99% sequence identity with the amino acid sequence of SEQ ID NO: 7;
   ii. a nucleotide sequence that has at least 15%, preferably at least 20, 30, 40, 50, 55, 60, 65, 70, 75, 80, 85, 90, 95, 96, 97, 98, 99% sequence identity with the nucleotide sequence of SEQ ID NO:8;
   iii. a nucleotide sequence the complementary strand of which hybridizes to a nucleic acid molecule of sequence of (i) or (ii); and
   iv. a nucleotide sequence which differs from the sequence of a nucleic acid molecule of (iii) due to the degeneracy of the genetic code,
e. a nucleotide sequence encoding alcohol dehydrogenase or acetaldehyde dehydrogenase, wherein said nucleotide sequence is selected from the group consisting of:
   i. a nucleotide sequence encoding an alcohol dehydrogenase or acetaldehyde dehydrogenase, said alcohol dehydrogenase or acetaldehyde dehydrogenase comprising an amino acid sequence that has at least 20%, preferably at least 30, 40, 50, 55, 60, 65, 70, 75, 80, 85, 90, 95, 96, 97, 98, 99% sequence identity with the amino acid sequence of SEQ ID NO: 9 and/or SEQ ID NO: 11, respectively
   ii. a nucleotide sequence comprising a nucleotide sequence that has at least 15%, preferably at least 20, 30, 40, 50, 55, 60, 65, 70, 75, 80, 85, 90, 95, 96, 97, 98, 99% sequence identity with the nucleotide sequence of SEQ ID NO:10 or SEQ ID NO: 12, respectively;
   iii. a nucleotide sequence the complementary strand of which hybridizes to a nucleic acid molecule of sequence of (i) or (ii); and
   iv. a nucleotide sequence which differs from the sequence of a nucleic acid molecule of (iii) due to the degeneracy of the genetic code, and,
f. a nucleotide sequence encoding NAD(P)H-dependent butanol dehydrogenase, wherein said nucleotide sequence is selected from the group consisting of:
   i. a nucleotide sequence encoding NAD(P)H-dependent butanol dehydrogenase, comprising an amino acid sequence that has at least 30%, preferably at least 40, 50, 55, 60, 65, 70, 75, 80, 85, 90, 95, 96, 97, 98, 99% sequence identity with the amino acid sequence of SEQ ID NO: 13 and/or SEQ ID NO: 15;
   ii. a nucleotide sequence comprising a nucleotide sequence that has at least 25%, preferably at least 30, 40, 50, 55, 60, 65, 70, 75, 80, 85, 90, 95, 96, 97, 98, 99% sequence identity with the nucleotide sequence of SEQ ID NO:14 and/or SEQ ID NO 16;
   iii. a nucleotide sequence the complementary strand of which hybridizes to a nucleic acid molecule of sequence of (i) or (ii); and,
   iv. a nucleotide sequence which differs from the sequence of a nucleic acid molecule of (iii) due to the degeneracy of the genetic code.

### Sequence Identity and Similarity

Sequence identity is herein defined as a relationship between two or more amino acid (polypeptide or protein) sequences or two or more nucleic acid (polynucleotide) sequences, as determined by comparing the sequences. Usually, sequence identities or similarities are compared over the whole length of the sequences compared. In the art, “identity” also means the degree of sequence relatedness between amino acid or nucleic acid sequences, as the case may be, as determined by the match between strings of such sequences. “Identity” and “similarity” can be readily calculated by various methods, known to those skilled in the art. Preferred methods to determine identity are designed to give the largest match between the sequences tested. Methods to determine identity and similarity are codified in publicly available computer programs. Preferred computer program methods to determine identity and similarity between two sequences include e.g. the BestFit, BLASTP, BLASTN, and FASTA (Altschul, S. F. et al., J. Mol. Biol. 215:403-410 (1990), publicly available from NCBI and other sources (BLAST Manual, Altschul, S., et al., NCBI NLM NIH Bethesda, Md. 20894). Preferred parameters for amino acid sequences comparison using BLASTP are gap open 10.0, gap extend 0.5, Blosum 62 matrix. Preferred parameters for nucleic acid sequences comparison using BLASTP are gap open 10.0, gap extend 0.5, DNA full matrix (DNA identity matrix).

### Hybridising Nucleic Acid Sequences

Nucleotide sequences encoding the enzymes expressed in the cell of the invention may also be defined by their capability to hybridise with the nucleotide sequences of SEQ ID NO.'s 2, 4, 6, 8, 10, 12, 14, 16 respectively, under moderate, or preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

The nucleotide sequences encoding an acetyl-CoA acetyltransferase, a 3-hydroxybutyryl-CoA dehydrogenase, a 3-hydroxybutyryl-CoA dehydratase, a butyryl-CoA dehydrogenase, an alcohol dehydrogenase or acetaldehyde dehydrogenase and/or NAD(P)H-dependent butanol dehydrogenase may be from prokaryotic or eukaryotic origin. A prokaryotic nucleotide sequence encoding an acetyl-CoA acetyltransferase may for instance be the thiL gene of Clostridium acetobutylicum as shown in SEQ ID. NO: 2. A prokaryotic nucleotide sequence encoding 3-hydroxybutyryl-CoA dehydrogenase may for instance be the hbd gene of Clostridium acetobutylicum as shown in sequence SEQ ID NO: 4. A prokaryotic nucleotide sequence encoding a 3-hydroxybutyryl-CoA dehydratase may for instance be the crt gene of Clostridium acetobutylicum as shown in sequence SEQ ID NO: 6. A prokaryotic nucleotide sequence encoding a butyryl-CoA dehydrogenase may for instance be the bcd gene of Clostridium acetobutylicum as shown in sequence SEQ ID NO: 8. A prokaryotic nucleotide sequence encoding alcohol dehydrogenase or acetaldehyde dehydrogenase may for instance be the adhE or adhE1 gene of Clostridium acetobutylicum as shown in sequence SEQ ID NO: 10 or SEQ ID NO: 12, respectively. A prokaryotic nucleotide sequence encoding NAD(P)H-dependent butanol dehydrogenase may for instance be the bdhA or bdhB gene of Clostridium acetobutylicum as shown in SEQ ID NO: 14 and SEQ ID NO: 16, respectively.

To increase the likelihood that the introduced enzymes are expressed in active form in a eukaryotic cell of the invention, the corresponding encoding nucleotide sequence may be adapted to optimise its codon usage to that of the chosen eukaryote host cell. The adaptiveness of the nucleotide sequences encoding the enzymes to the codon usage of the chosen host cell may be expressed as codon adaptation index (CAI). The codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al., 2003, Nucleic Acids Res. 31(8):2242-51). An adapted nucleotide sequence preferably has a CAI of at least 0.2, 0.3, 0.4, 0.5, 0.6 or 0.7.

In a preferred embodiment the eukaryotic cell according to the present invention is genetically modified with (a) nucleotide sequence(s) which is (are) adapted to the codon usage of the eukaryotic cell using codon pair optimisation technology as disclosed in PCT/EP2007/05594. Codon-pair optimisation is a method for producing a polypeptide in a host cell, wherein the nucleotide sequences encoding the polypeptide have been modified with respect to their codon-usage, in particular the codon-pairs that are used, to obtain improved expression of the nucleotide sequence encoding the polypeptide and/or improved production of the polypeptide. Codon pairs are defined as a set of two subsequent triplets (codons) in a coding sequence.

It was surprisingly found that when the nucleotide sequences in the transformed eukaryotic cell were adapted to the chosen eukaryotic cell using the codon pair optimization method, the amount of butanol produced by the eukaryotic cell was increased compared to the eukaryotic cell that was transformed with nucleotide sequences that were not codon pair optimized.

Further improvement of the activity of the enzymes in vivo in a eukaryotic host cell of the invention, can be obtained by well-known methods like error prone PCR or directed evolution. A preferred method of directed evolution is described in WO03010183 and WO03010311.

The eukaryotic cell according to the present invention may be any suitable host cell, preferably from microbial origin. Preferably, the host cell is a yeast or a filamentous fungus. More preferably, the host cell belongs to one of the genera Saccharomyces, Aspergillus, Penicillium, Pichia, Kluyveromyces, Yarrowia, Candida, Hansenula, Humicola, Torulaspora, Trichosporon, Brettanomyces, Pachysolen or Yamadazyma. A more preferred host cell belongs to the species Aspergillus niger, Penicillium chrysogenum, Pichia stipidis, Kluyveromyces marxianus, K. lactis, K. thermotolerans, Yarrowia lipolytica, Candida sonorensis, C. glabrata, Hansenula polymorpha, Torulaspora delbrueckii, Brettanomyces bruxellensis, Zygosaccharomyces bailii, Saccharomyces uvarum, Saccharomyces bayanus or Saccharomyces cerevisiae species. Preferably, the eukaryotic cell is a Saccharomyces cerevisiae.

Preferably, the eukaryotic cell according to the invention is a yeast, preferably Saccharomyces cerevisiae, comprising one or more of the genes selected from the group consisting of SEQ ID NO. 17, SEQ ID NO. 18, SEQ ID NO. 19, SEQ ID NO. 20, SEQ ID NO. 21 or SEQ ID NO 22, and SEQ ID NO 23 or SEQ ID NO 24.

The nucleotide sequences encoding the enzymes that produce acetoacetyl-CoA, 3-hydroxybutyryl-CoA, crotonyl-CoA, butyryl-CoA, butyrylaldehyde and butanol, may be ligated into a nucleic acid construct to facilitate the transformation of the eukaryotic cell according to the present invention. A nucleic acid construct may be a plasmid carrying the genes encoding all six enzymes of the butanol metabolic pathway as described above, or a nucleic acid construct comprises two or three plasmids carrying each three or two genes, respectively, encoding the six enzymes of the butanol pathway distributed in any appropriate way. Any suitable plasmid may be used, for instance a low copy plasmid or a high copy plasmid. It may be possible that the enzymes selected from the group consisting of acetyl-CoA acetyltransferase, 3-hydroxybutyryl-CoA dehydrogenase, 3-hydroxybutyryl-CoA dehydratase, butyryl-CoA dehydrogenase, alcohol dehydrogenase or acetaldehyde dehydrogenase, and NAD(P)H-dependent butanol dehydrogenase are native to the host cell and that transformation with one or more of the nucleotide sequences encoding these enzymes may not be required to confer the host cell the ability to produce butanol. Further improvement of butanol production by the host cell may be obtained by classical strain improvement.

The nucleic acid construct may be maintained episomally and thus comprise a sequence for autonomous replication, such as an autosomal replication sequence sequence. If the host cell is of fungal origin, a suitable episomal nucleic acid construct may e.g. be based on the yeast 2μ or pKD1 plasmids (Gleer et al., 1991, Biotechnology 9: 968-975), or the AMA plasmids (Fierro et al., 1995, Curr Genet. 29:482-489). Alternatively, each nucleic acid construct may be integrated in one or more copies into the genome of the host cell. Integration into the host cell's genome may occur at random by non-homologous recombination but preferably the nucleic acid construct may be integrated into the host cell's genome by homologous recombination as is well known in the art (see e.g. WO90/14423, EP-A-0481008, EP-A-0635 574 and U.S. Pat. No. 6,265,186).

Optionally, a selectable marker may be present in the nucleic acid construct. As used herein, the term “marker” refers to a gene encoding a trait or a phenotype which permits the selection of, or the screening for, a host cell containing the marker. The marker gene may be an antibiotic resistance gene whereby the appropriate antibiotic can be used to select for transformed cells from among cells that are not transformed. Preferably however, non-antibiotic resistance markers are used, such as auxotrophic markers (URA3, TRP1, LEU2). The host cells transformed with the nucleic acid constructs may be marker gene free. Methods for constructing recombinant marker gene free microbial host cells are disclosed in EP-A-0 635 574 and are based on the use of bidirectional markers. Alternatively, a screenable marker such as Green Fluorescent Protein, lacZ, luciferase, chloramphenicol acetyltransferase, beta-glucuronidase may be incorporated into the nucleic acid constructs of the invention allowing to screen for transformed cells. A preferred marker-free method for the introduction of heterologous polynucleotides is described in WO0540186.

In a preferred embodiment, the nucleotide sequences encoding the enzymes that produce acetoacetyl-CoA, 3-hydroxybutyryl-CoA, crotonyl-CoA, butyryl-CoA butyrylaldehyde and butanol, for instance the enzyme as defined herein, are each operably linked to a promoter that causes sufficient expression of the corresponding nucleotide sequences in the eukaryotic cell according to the present invention to confer to the cell the ability to produce butanol.

As used herein, the term “operably linked” refers to a linkage of polynucleotide elements (or coding sequences or nucleic acid sequence) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skilled in the art to act directly or indirectly to regulate the amount of transcription from the promoter. A “constitutive” promoter is a promoter that is active under most environmental and developmental conditions. An “inducible” promoter is a promoter that is active under environmental or developmental regulation.

The promoter that could be used to achieve the expression of the nucleotide sequences coding for an enzyme as defined herein above, may be not native to the nucleotide sequence coding for the enzyme to be expressed, i.e. a promoter that is heterologous to the nucleotide sequence (coding sequence) to which it is operably linked. Preferably, the promoter is homologous, i.e. endogenous to the host cell

Suitable promoters in eukaryotic host cells may be GAL7, GAL10, or GAL 1, CYC1, HIS3, ADH1, PGL, PHO5, GAPDH, ADC1, TRP1, URA3, LEU2, ENO, TPI, and AOX1. Other suitable promoters include PDC, GPD1, PGK1, TEF1, and TDH.

Any terminator, which is functional in the cell, may be used in the present invention. Preferred terminators are obtained from natural genes of the host cell. Suitable terminator sequences are well known in the art. Preferably, such terminators are combined with mutations that prevent nonsense mediated mRNA decay in the host cell of the invention (see for example: Shirley et al., 2002, Genetics 161:1465-1482).

Preferred promoters and terminators are shown in SEQ ID NO. 25 to 30.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but have been obtained from another cell or synthetically or recombinantly produced.

One or more enzymes of the butanol pathway as described above may be overexpressed to achieve a sufficient butanol production by the cell.

There are various means available in the art for overexpression of enzymes in the host cells of the invention. In particular, an enzyme may be overexpressed by increasing the copy number of the gene coding for the enzyme in the host cell, e.g. by integrating additional copies of the gene in the host cell's genome.

A preferred host cell according to the present invention may be a eukaryotic cell which is naturally capable of alcoholic fermentation, for instance anaerobic alcohol fermentation, in particular ethanol fermentation. A group of eukaryotic cells which is able to produce ethanol is for instance yeast, such as Saccharomyces cerevisiae. If the eukaryotic cell is capable of ethanol fermentation, it may be preferred that one or more genes encoding pyruvate decarboxylase is/are knocked out, in order to shift the metabolism to the butanol pathway.

To further increase the butanol production, it may be preferred to increase the cytosolic acetyl CoA pool in the eukaryotic host cell by growing the eukaryotic cell in the presence of a mixture of fermentable carbon source (eg. glucose or galactose) and acetate, or acetate sources such as fatty acids, in order to provide the cell with sufficient cytosolic acetyl-CoA.

Preferably, the host cell according to the present invention further has a high tolerance to alcohols, such as ethanol, propanol, butanol, isopropanol, isobutanol, isoamyl alcohol, pentanol, hexanol, heptanol, or octanol. A high alcohol tolerance may be naturally present in the host cell or may be introduced or modified by genetic modification, which may include classical strain improvement techniques or directed evolution.

A preferred transformed eukaryotic cell according to the present invention may be able to grow on any suitable carbon source known in the art and convert it to butanol. The transformed eukaryotic host cell may be able to convert directly plant biomass, celluloses, hemicelluloses, pectines, rhamnose, galactose, fucose, maltose, maltodextrines, ribose, ribulose, or starch, starch derivatives, sucrose, lactose and glycerol. Hence, a preferred host organism expresses enzymes such as cellulases (endocellulases and exocellulases) and hemicellulases (e.g. endo- and exo-xylanases, arabinases) necessary for the conversion of cellulose into glucose monomers and hemicellulose into xylose and arabinose monomers, pectinases able to convert pectines into glucuronic acid and galacturonic acid or amylases to convert starch into glucose monomers. Preferably, the host cell is able to convert a carbon source selected from the group consisting of glucose, xylose, arabinose, sucrose, lactose and glycerol. The host cell may for instance be a eukaryotic host cell as described in WO03/062430, WO06/009434, EP1499708B1, WO2006096130 or WO04/099381.

In a further aspect, the present invention relates to a process for the production of butanol comprising fermenting a transformed eukaryotic cell according to the present invention in a suitable fermentation medium, and optionally recovering the butanol.

The fermentation medium used in the process for the production of butanol may be any suitable fermentation medium which allows growth of a particular eukaryotic host cell. The essential elements of the fermentation medium are known to the person skilled in the art and may be adapted to the host cell selected.

Preferably, the fermentation medium comprises acetate. It was surprisingly found that when the eukaryotic cell was grown in the presence of acetate, an increased amount of butanol was produced compared to a cell which was grown in the absence of acetate. The concentration of acetate in the fermentation medium is between 0.5 and 5 g/l, preferably between, 1 and 4 g/l, preferably between 1.5 and 3.5 g/l.

Preferably, the fermentation medium comprises a carbon source selected from the group consisting of plant biomass, celluloses, hemicelluloses, pectines, rhamnose, galactose, fucose, fructose, maltose, maltodextrines, ribose, ribulose, or starch, starch derivatives, sucrose, lactose, fatty acids, triglycerides and glycerol. Preferably, the fermentation medium also comprises a nitrogen source such as ureum, or an ammonium salt such as ammonium sulphate, ammonium chloride, ammoniumnitrate or ammonium phosphate.

The fermentation process according to the present invention may be carried out in batch, fed-batch or continuous mode. A separate hydrolysis and fermentation (SHF) process or a simultaneous saccharification and fermentation (SSF) process may also be applied. A combination of these fermentation process modes may also be possible for optimal productivity. A SSF process may be particularly attractive if starch, cellulose, hemicelluose or pectin is used as a carbon source in the fermentation process, where it may be necessary to add hydrolytic enzymes, such as cellulases, hemicellulases or pectinases to hydrolyse the substrate.

The transformed eukaryotic cell used in the process for the production of butanol may be any suitable host cell as defined herein above. It was found advantageous to use a transformed eukaryotic cell according to the invention in the process for the production of butanol, because most eukaryotic cells do not require sterile conditions for propagation and are insensitive to bacteriophage infections. In addition, eukaryotic host cells can be grown at low pH to prevent bacterial contamination.

Preferably, the eukaryotic cell according to the present invention is a facultative anaerobic microorganism. A facultative anaerobic micro organism is preferred because a facultative microorganism can be propagated aerobically to a high cell concentration and butanol can be produced subsequently under anaerobic conditions. This anaerobic phase can then be carried out at high cell density which reduces the fermentation volume required substantially, and minimizes the risk of contamination with aerobic microorganisms.

The fermentation process for the production of butanol according to the present invention may be an aerobic or an anaerobic fermentation process.

An anaerobic fermentation process is herein defined as a fermentation process run in the absence of oxygen or in which substantially no oxygen is consumed, preferably less than 5, 2.5 or 1 mmol/L/h, and wherein organic molecules serve as both electron donor and electron acceptors. The fermentation process according to the present invention may also first be run under aerobic conditions and subsequently under anaerobic conditions.

The fermentation process may also be run under oxygen-limited, or micro-aerobical, conditions. Alternatively, the fermentation process may first be run under aerobic conditions and subsequently under oxygen-limited conditions. An oxygen-limited fermentation process is a process in which the oxygen consumption is limited by the oxygen transfer from the gas to the liquid. The degree of oxygen limitation is determined by the amount and composition of the ingoing gasflow as well as the actual mixing/mass transfer properties of the fermentation equipment used. Preferably, in a process under oxygen-limited conditions, the rate of oxygen consumption is at least 5.5, more preferably at least 6 and even more preferably at least 7 mmol/L/h.

The production of butanol in the process according to the present invention may occur during the growth phase of the host cell, during the stationary (steady state) phase or during both phases. It may be possible to run the fermentation process at different temperatures.

The process for the production of butanol is preferably run at a temperature which is optimal for the eukaryotic cell. The optimum growth temperature may differ for each transformed eukaryotic cell and is known to the person skilled in the art. The optimum temperature might be higher than optimal for wild type organisms to grow the organism efficiently under non-sterile conditions under minimal infection sensitivity and lowest cooling cost.

The optimum temperature for growth of the transformed eukaryotic cell may be above 20° C., 22° C., 25° C., 28° C., or above 30° C., 35° C., or above 37° C., 40° C., 42° C., and preferably below 45° C. During the production phase of butanol however, the optimum temperature might be lower than average in order to optimize biomass stability and reduce butanol solubility. The temperature during this phase may be below 45° C., for instance below 42° C., 40° C., 37° C., for instance below 35° C., 30° C., or below 28° C., 25° C., 22° C. or below 20° C. preferably above 15° C.

The process for the production of butanol according to the present invention may be carried out at any suitable pH value. If the transformed eukaryotic cell is yeast, the pH in the fermentation medium preferably has a value of below 6, preferably below 5.5, preferably below 5, preferably below 4.5, preferably below 4, preferably below pH 3.5 or below pH 3.0, or below pH 2.5, preferably above pH 2. An advantage of carrying out the fermentation at these low pH values is that growth of contaminant bacteria in the fermentation medium may be prevented.

Recovery of butanol from the fermentation medium may be performed by known methods in the art, for instance by distillation, vacuum extraction, solvent extraction, or pervaporation.

It was found that the process for the production of butanol according to the invention results in a concentration of above 5 mg/l fermentation broth, preferably above 10 mg/l, preferably above 20 mg/l, preferably above 30 mg/l fermentation broth, preferably above 40 mg/l, more preferably above 50 mg/l, preferably above 60 mg/l, preferably above 70, preferably above 80 mg/l, preferably above 100 mg/l, preferably above 1 g/l, preferably above 5 g/l, preferably above 10 g/l, but usually below 70 g/l.

The present invention also relates to a fermentation broth comprising butanol obtainable by the process according to the present invention. It was found that the fermentation broth comprises no or a low concentration of butyrate and acetone.

The butanol produced by the fermentation process according to the present invention may be used in any application known for butanol. It may for instance be used as a fuel, for instance as additive to diesel or gasoline. Alternatively fermentatively produced butanol may be converted to butylacrylate by known methods in the art.

Genetic Modifications

Standard genetic techniques, such as overexpression of enzymes in the host cells, as well as for additional genetic modification of host cells, are known methods in the art, such as described in Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation and genetic modification of fungal host cells are known from e.g. EP-A-0 635 574, WO 98/46772, WO 99/60102 and WO 00/37671.

The following examples are for illustrative purposes only and are not to be construed as limiting the invention.

### EXAMPLES

### General

oligonucleotides were synthesized by Invitrogen (Carlsbad Calif., US).
DNA sequencing was performed at SEQLAB (Göttingen, Germany) or by Baseclear (Leiden, The Netherlands)
Restriction enzymes were supplied by Invitrogen or New England Biolabs.
Used strains: Escherichia coli DH10B electromax competent cells (Invitrogen). Protocol is delivered by manufacturer.
SDS-PAGE system (Invitrogen)
NuPAGE Novex Bis-Tris Gels (Invitrogen). SimplyBlue SafeStain Microwave protocol

### Example 1

### Cloning of the Butanol Biosynthesis Route in Saccharomyces cerevisiae by Homologous Recombination and Production of Butanol

1.1. Genes and Constructs

For introduction of the butanol pathway in S. cerevisiae, 8 Clostridium acetobutylicum genes are cloned in total:
thiL encoding acetyl CoA-acetyltransfrase [E.C.2.3.1.9] (SEQ ID. NO:2)
hbd encoding 3-hydroxybutyryl-CoA dehydrogenase [E.C.1.1.1.57] (SEQ ID NO:4)
crt encoding crotonase or 3-hydroxybutyryl-CoA dehydratase [E.C.4.2.1.55] (SEQ ID NO:6)
bcd encoding butyryl-CoA dehydrogenase [E.C.1.3.99.2] (SEQ ID NO: 8)
adhE or adhE1 both encoding alcohol dehydrogenase or acetaldehyde dehydrogenase [E.C.1.2.1.10] (SEQ ID NO: 10 and SEQ ID NO:12)
bdhA, bdhB both encoding respectively NADH-dependent butanol dehydrogenase A and B [E.C.:1.1.1.-] (SEQ ID NO: 14 and 16)

The expression constructs are synthesized at DNA2.0 (Menlo Park Calif., USA). Two high-copy expression shuttle vectors, pRS425 and pRS426 derived (Sirkoski R. S. and Hieter P. Genetics, 1989, 122(1):19-27), are created each expressing 3 of the butanol biosynthesis genes.

All synthesized constructs contain 40 bp homologous flanks for tripartite homologous recombination as described by Raymon C. K. et al Biotechniques (1999) 26:134-141. The thiL gene and the hbd gene are synthesized as one fragment expressed from the bi-directional GAL1-10 promoter and terminated by the GAL1-10 terminators. The crt and bcd are expressed from a similar construct. The adhE and adhE1 genes are synthesized between the GAL7 promoter and terminator as well as bdhA and bdhB, resulting in 4 different constructs.

1.2. Transformation of S. cerevisiae

The first two expression constructs are created by tripartite in vivo homologous recombination in S. cerevisiae CEN.PK102-3A (ura3-52 and leu2-3) of the thiL/hbd construct with the adhE or adhE1 expression construct and the linearized pRS425 expression vector (LEU2) resulting in pRS425THE and pRS425THE1.

The second two expression vectors are created by tripartite in vivo homologous recombination in CEN.PK102-3A of the crt/bcd expression construct and the bdhA or bdhB expression construct with the linearized pRS426 expression vector (URA3), resulting in pRS426CBA and pRS426CBB, respectively.

Each plasmid contains 3 genes behind galactose inducible promoters.

The plasmids are isolated from the transformed S. cerevisiae strains and E. coli DH10b is transformed with the expression vectors to select and check the correct plasmids.

CEN.PK102-3A is transformed with a) pRS425THE and pRS426CBA, b) pRS425THE and pRS426CBB, c) pRS425THE1 and pRS426CBA, d) pRS425THE1 and pRS426CBB. Transformants are plated on Yeast Nitrogen Base (YNB) w/o AA (Difco)+2% glucose. In total 10 transformants of each plasmid combination are inoculated in YNB w/o AA (Difco)+0.1% glucose+2% galactose and grown under aerobic conditions in shake flasks, anaerobic or oxygen-limited conditions in 10 ml cultures. The medium for anaerobic cultivation is supplemented with 0.01 g/l ergosterol and 0.42 g/l Tween 80 dissolved in ethanol (Andreasen and Stier, 1953, J. cell. Physiol, 41, 23-36; Andreasen and Stier, 1954, J. Cell. Physiol, 43: 271-281). All yeast cultures are grown at 30° C. After growth and induction overnight the cells are spun down and the butanol concentration is measured in the supernatant by HPLC as described below.

1.3. Butanol Analysis by HPLC

HPLC analysis: pre-column: Biorad Microguard Cation H+ cartridge. Column: Biorad Aminex HPX-87H. Mobile phase: 0.01N H2SO4. Precipitation reagent: 3.3N HClO4. RI detection: Waters 410 differential refractometer.

### Example 2

### Cloning of the Butanol Biosynthesis Route in Saccharomyces cerevisiae Using Restriction Enzymes and Production of Butanol Using Codon-Pair Optimized Genes

2.1. Genes and Constructs

The codon-pair method as disclosed in PCT/EP2007/05594 was applied to the 8 native genes given in Example 1 under paragraph 1.1. for expression in S. cerevisiae. This resulted in 8 codon-pair optimized variants:

SEQ ID NO. 17: Codon pair optimised (CPO) thiL gene

SEQ ID NO. 18: Codon pair optimised hbd gene

SEQ ID NO. 19: Codon pair optimised crt gene (counterclockwise)

SEQ ID NO. 20: Codon pair optimised bcd gene

SEQ ID NO. 21: Codon pair optimised adhE gene

SEQ ID NO 22: Codon pair optimised adhE1 gene

SEQ ID NO 23: Codon pair optimised bdhA gene

SEQ ID NO 24: Codon pair optimised bdhB gene

The 8 designed codon pair optimised genes were synthesised at DNA2.0 (Menlo Park Calif., USA). The codon-pair optimised genes were used to make the expression constructs as described below.

2.2. Transformation of S. cerevisiae

The first two expression constructs were created after an ApaI/NotI restriction enzyme double digest of the pRS415 vector (LEU) and subsequently ligating in this vector an ApaI/AscI restriction fragment consisting of either adhE or adhE1 gene combined with an AscI/NotI restriction fragment containing the thiL/hbd fragment. After this triple ligation the ligation mix was used for transformation of E. coli DH10B (Invitrogen) resulting in constructs pRS415THE and pRS415THE1, respectively. These constructs were subsequently used for transformation in S. cerevisiae CEN.PK102-3A (ura3-52 and leu2-3).

The second two expression vectors were created after a BamHI/NotI restriction enzyme double digest of the pRS416 vector (URA) and subsequently ligating in this vector a BamHI/AscI restriction fragment consisting of either bdhA or bdhB gene combined with an AscI/NotI restriction fragment containing the crt/bcd fragment. After this triple ligation, the ligation mix was used for transformation of E. coli DH10B (Invitrogen) resulting in constructs pRS416CBA and pRS416CBB, respectively. These constructs were subsequently used for transformation in S. cerevisiae CEN.PK102-3A (ura3-52 and leu2-3).

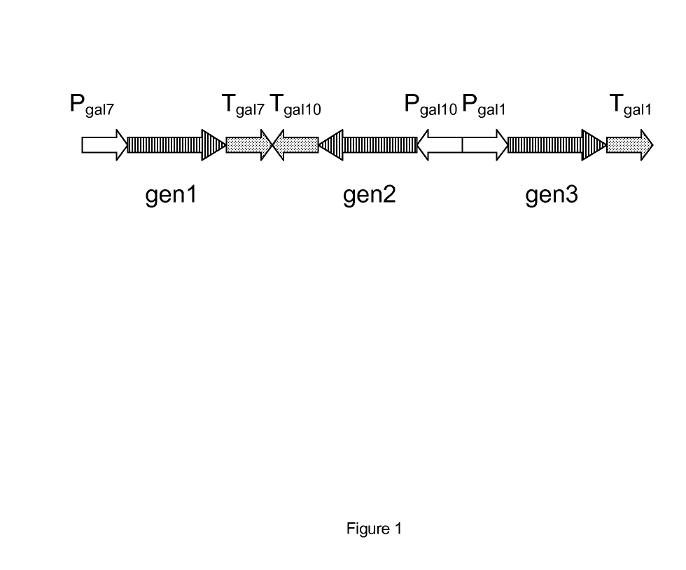

Each plasmid contained 3 genes behind galactose inducible promoters and terminators. A schematic presentation of the constructs is shown in FIG. 1. The sequence listings of the promoters and terminators are as follows: SEQ ID NO 25: GAL7 promoter; SEQ ID NO. 26: GAL 7 terminator; SEQ ID NO 27: GAL 10 terminator, counterclockwise; SEQ ID NO 28: GAL 10 promoter, counterclockwise; SEQ ID NO 29: GAL 1 promoter; SEQ ID NO 30: GAL 1 terminator

S. cerevisiae CEN.PK102-3A was transformed with a) pRS415THE and pRS416CBA, b) pRS415THE and pRS416CBB, c) pRS415THE1 and pRS416CBA, or d) pRS415THE1 and pRS416CBB. Transformants were plated on Yeast Nitrogen Base (YNB) w/o AA (Difco)+2% glucose. In total 10 transformants of each plasmid combination were inoculated in YNB w/o AA (Difco)+0.1% glucose+2% galactose and under aerobic conditions. Subsequently the transformed yeasts were grown microaerobically in 10 ml cultures in flasks which were closed with rubber stoppers and aluminium caps. All yeast cultures were grown at 25° C. After induction overnight, aliquots of the cultures were removed after 40, 48 and 64 hours of cultivation. The cells were spun down and the butanol concentration was measured in the supernatant by Headspace Gaschromatography (HS-GC) as described below.

2.3. Butanol Analysis by HS-GC

Samples were analysed on a HS-GC equipped with a flame ionisation detector and an automatic injection system. Column J&W DB-1 length 30 m, id 0.53 mm, df 5 μm. The following conditions were used: helium as carrier gas with a flow rate of 5 ml/min. Column temperature was set at 110° C. The injector was set at 140° C. and the detector performed at 300° C. The data were achieved using Chromeleon software. Samples were heated at 60° C. for 20 min in the headspace sampler. One ml of the headspace volatiles were automatically injected on the column.

All different transformants comprising either the codon optimised adhE and bdhA or bdhA, or adhE1 and bdhA or bdhB were able to produce butanol. The butanol concentration in the supernatant was between 5-10 mg butanol/l after 40 h of cultivation, 8-13 mg butanol/l after 48 h of cultivation, and 15 and 20 mg butanol/l after 64 h of cultivation. A S. cerevisiae strain comprising non-codon pair optimised genes produced 0.1-2 mg/l after 64 h of cultivation.

### Example 3

### Effect of Acetate on Butanol Yield

The effect of the presence of acetate in the fermentation medium on the yield of butanol was determined with the butanol producing yeast strain CEN.PK102-3A strain transformed with pRS425THE and pRS426CBB as described in example 2. This yeast strain was inoculated in Verduyn medium (Verduyn, C., Postma, E., Scheffers W. A., van Dijken, J. P. (1992), Yeast 8, 501-517), which was adjusted as follows: ammonium sulphate was replaced with ureum (2 g/l), galactose (40 g/l) was the sole carbon source and the medium was supplemented with 2 g/l potassium acetate, 0.01 g/l ergosterol and 0.42 g/l tween 80 dissolved in ethanol (Andreasen and Stier, 1953, J. cell. Physiol, 41, 23-36; Andreasen and Stier, 1954, J. Cell. Physiol, 43: 271-281). The reference cultures did not contain 2 g/l potassium acetate. Cells were grown micro aerobically in 50 ml medium in flasks which were closed with rubber stoppers and aluminium caps. The flasks were shaken gently at 25° C. At an optical density of 1.5 at 600 nm (after 72 hours) the cells were spun down and the butanol concentration was measured in the supernatant by HS-GC as described example 2.

The yield of butanol in cultures comprising acetate was 20 mg/l. The yield of butanol in cultures that did not comprise acetate was 13 mg/l.

1. A transformed eukaryotic cell comprising one or more nucleotide sequencers) encoding acetyl-CoA acetyltransferase, 3-hydroxybutyryl-CoA dehydrogenase, 3-hydroxybutyryl-CoA dehydratase, butyryl-CoA dehydrogenase, alcohol dehydrogenase or acetaldehyde dehydrogenase and/or NAD(P)H-dependent butanol dehydrogenase, whereby the nucleotide sequence(s) upon transformation of the cell confer(s) on the cell the ability to produce butanol.

2. A cell according to claim 1, wherein the nucleotide sequencers) has (have) been adapted to the codon usage of the eukaryotic cell using codon pair optimisation.

3. A eukaryotic cell according to claim 1, which expresses one or more nucleotide sequencers) selected from the group consisting of:
a. a nucleotide sequence encoding an acetyl-CoA acetyltransferase, wherein said nucleotide sequence is selected from the group consisting of:
i. a nucleotide sequence encoding an acetyl-CoA acetyltransferase, said acetyl CoA acetyltransferase comprising an amino acid sequence that has at least 20% sequence identity with the amino acid sequence of SEQ ID NO:1;
ii. a nucleotide sequence that has at least 15% sequence identity with the nucleotide sequence of SEQ ID NO:2;
iii. a nucleotide sequence, the complementary strand of which hybridizes to a nucleotide sequence of (i) or (ii); and
iv. a nucleotide sequence which differs from the nucleotide sequence of a (iii) due to the degeneracy of the genetic code;
b. a nucleotide sequence encoding a 3-hydroxybutyryl-CoA dehydrogenase, wherein said nucleotide sequence is selected from the group consisting of:
i. a nucleotide sequence encoding a 3-hydroxybutyryl-CoA dehydrogenase, said 3-hydroxybutyryl-CoA dehydrogenase comprising an amino acid sequence that has at least 25% sequence identity with the amino acid sequence of SEQ ID NO: 3;
ii. a nucleotide sequence that has at least 20% sequence identity with the nucleotide sequence of SEQ ID NO:4;
iii. a nucleotide sequence, the complementary strand of which hybridizes to a nucleotide sequence of (i) or (ii); and
iv. a nucleotide sequence which differs from the nucleotide sequence of (iii) due to the degeneracy of the genetic code;
c. a nucleotide sequence encoding 3-hydroxybutyryl-CoA dehydratase, wherein said nucleotide sequence is selected from the group consisting of:
i. a nucleotide sequence encoding a 3-hydroxybutyryl-CoA dehydratase, said 3-hydroxybutyryl-CoA dehydratase comprising an amino acid sequence that has at least 30% sequence identity with the amino acid sequence of SEQ ID NO: 5;
ii. a nucleotide sequence comprising a nucleotide sequence that has at least 25% sequence identity with the nucleotide sequence of SEQ ID NO:6;
iii. a nucleotide sequence the complementary strand of which hybridizes to a nucleotide sequence of (i) or (ii); and
iv. a nucleotide sequence which differs from the nucleotide sequence of a (iii) due to the degeneracy of the genetic code,
d. a nucleotide sequence encoding butyryl-CoA dehydrogenase, wherein said nucleotide sequence is selected from the group consisting of:
i. a nucleotide sequence encoding a butyryl-CoA dehydrogenase, said butyryl-CoA dehydrogenase comprising an amino acid sequence that has at least 20% sequence identity with the amino acid sequence of SEQ ID NO: 7;
ii. a nucleotide sequence that has at least 15% sequence identity with the nucleotide sequence of SEQ ID NO:8;
iii. a nucleotide sequence, the complementary strand of which hybridizes to a nucleic acid molecule of sequence of (i) or (ii); and
iv. a nucleotide sequence which differs from the nucleotide sequence of (iii) due to the degeneracy of the genetic code;
e. a nucleotide sequence encoding alcohol dehydrogenase or acetaldehyde dehydrogenase, wherein said nucleotide sequence is selected from the group consisting of:
i. a nucleotide sequence encoding an alcohol dehydrogenase or acetaldehyde dehydrogenase, said alcohol dehydrogenase or acetaldehyde dehydrogenase comprising an amino acid sequence that has at least 20% sequence identity with the amino acid sequence of SEQ ID NO: 9 and/or SEQ ID NO: 11, respectively
ii. a nucleotide sequence comprising a nucleotide sequence that has at least 15% sequence identity with the nucleotide sequence of SEQ ID NO:10 or SEQ ID NO: 12 respectively;
iii. a nucleotide sequence, the complementary strand of which hybridizes to a nucleotide sequence of (i) or (ii); and
iv. a nucleotide sequence which differs from the nucleotide sequence of (iii) due to the degeneracy of the genetic code; and
f. a nucleotide sequence encoding NAD(P)H-dependent butanol dehydrogenase, wherein said nucleotide sequence is selected from the group consisting of:
i. a nucleotide sequence encoding NAD(P)H-dependent butanol dehydrogenase, comprising an amino acid sequence that has at least 30% sequence identity with the amino acid sequence of SEQ ID NO: 13 and/or SEQ ID NO: 15;
ii. a nucleotide sequence comprising a nucleotide sequence that has at least 25% sequence identity with the nucleotide sequence of SEQ ID NO:14 and/or SEQ ID NO 16;
iii. a nucleotide sequence, the complementary strand of which hybridizes to a nucleotide sequence of (i) or (ii); and
iv. a nucleotide sequence which differs from the nucleotide sequence of (iii) due to the degeneracy of the genetic code.

4. A cell according to claim 1, wherein the cell is a Saccharomyces cerevisiae which comprises heterologous nucleotide sequences encoding acetyl-CoA acetyltransferase, 3-hydroxybutyryl-CoA dehydrogenase, 3-hydroxybutyryl-CoA dehydratase, butyryl-CoA dehydrogenase, alcohol dehydrogenase or acetaldehyde dehydrogenase and NAD(P)H-dependent butanol dehydrogenase.

5. A cell according to claim 1, which is a Saccharomyces cerevisiae comprising one or more nucleotide sequence(s) selected from the group consisting of SEQ ID NO. 17, SEQ ID NO. 18, SEQ ID NO. 19, SEQ ID NO. 20, SEQ ID NO. 21 or SEQ ID NO 22, and SEQ ID NO 23 or SEQ ID NO 24.

6. A cell according to claim 1, wherein one or more gene(s) encoding pyruvate decarboxylase is(are) knocked out.

7. A cell according to claim 1, which is able to convert a carbon source selected from the group consisting of starch, pectines, rhamnose, galactose, fucose, fructose, maltose, maltodextrines, ribose, ribulose, cellulose, hemicellulose, glucose, xylose, arabinose, sucrose, lactose, fatty acids, triglycerides and glycerol.

8. Process for the production of butanol comprising fermenting a transformed eukaryotic cell as defined in claim 1 in a suitable fermentation medium, and optionally recovering butanol.

9. Process according to claim 8, which is carried out at a pH of below 5.

10. Process according to claim 8, characterised in that the fermentation medium comprises acetate.

11. A fermentation broth comprising butanol obtainable by the process according to claim 8.
